(19) United States
(12) Patent Application Publication
(10) Pub. No.: US 2024/0145027 A1
(43) Pub. Date: May 2, 2024

(54) A METHOD AND SYSTEM TO BUILD AN IR FINGERPRINT DATABASE FOR THE STRUCTURAL IDENTIFICATION OF BIOMOLECULES

(71) Applicant: ECOLE POLYTECHNIQUE FEDERALE DE LAUSANNE (EPFL), Lausanne (CH)

(72) Inventors: Priyanka BANSAL, Crissier (CH); Ahmed BEN FALEH, Renens (CH); Robert PELLEGRINELLI, Lausanne (CH); Stephan WARNKE, Preverenges (CH); Thomas RIZZO, Denens (CH)

(21) Appl. No.: 18/567,573

(22) PCT Filed: Jun. 1, 2022
(86) PCT No.: PCT/IB2022/055139

§ 371 Date: Dec. 6, 2023

(30) Foreign Application Priority Data
Jun. 8, 2021 (WO) PCT/IB2021/055037

(51) Int. Cl. G16B 15/00; G01N 21/35; G01N 27/623; G01N 33/68; G16B 40/10; H01J 49/04

(52) U.S. Cl. CPC G16B 15/00; G01N 21/35; G01N 27/623; G01N 33/6851; G16B 40/10; H01J 49/0481; G01N 2201/06113

(57) ABSTRACT

A method, system, and computer-readable medium for identifying and creating a database of isomers and isobars of molecules including the steps of performing isomer or isobar separation on molecules to obtain separate isomeric or isobaric molecules, measuring mass-to-charge ratios (m/z) to obtain IR fingerprints of the separate isomeric or isobaric molecules, and storing first data on the mass-to-charge ratios (m/z) and/or the IR fingerprints of the separate isomeric or isobaric molecules to a database.

### CROSS REFERENCE TO RELATED APPLICATIONS

The present patent application claims priority to International patent application with the Serial No. PCT/IB2021/055037 filed on Jun. 8, 2021, this reference herewith incorporated by reference in its entirety.

### FIELD OF THE INVENTION

The present invention provides a method to identify isomers and isobars of biomolecules, such as glycans, or metabolites, using a database approach. The method is capable to characterize unknown isomeric structures with respect to their regio- and stereochemistry. At the same time, the method can provide a means to build a database for identification of such compounds with a reduced need for isomerically pure analytical standards.

### BACKGROUND

Identification and differentiation of isomeric molecules is of general analytical interest. As biomarkers for disease, metabolites and oligosaccharides (or glycans) often need to be distinguished among a pool of their respective isomeric structures. For example, the efficacy, toxicity, and shelf life of an antibody drug is largely influenced by its glycan shield, which is why a precise characterization of the glycan content is required for approval of such a drug. The structural complexity of metabolites and glycans give rise to many types of regio- and stereoisomers that pose a challenge to state-of-the-art analytical techniques. For example, a particular class of glycan isomers are structural- or positional isomers, which are characterized by having identical monosaccharide constituents that form their glycosidic linkage at different positions within the molecule. If multiple branches are present in a glycan, monosaccharides can reside on either branch, and each isomer will exhibit different properties. Separation or identification of such isomers is currently cumbersome, if not impossible. By its very nature, a simple mass measurement cannot distinguish isomers and thus needs to be combined with either fragmentation techniques (tandem mass spectrometry (MSⁿ)) or separation techniques such as liquid chromatography (LC), gas chromatography (GC), or ion mobility spectrometry (IMS). While LC-MS techniques can be considered a workhorse in the bioanalytical field, they can be relatively slow, often involving additional sample derivatization steps. Moreover, not all types of isomers can be separated or identified using this technique. On the other hand, nuclear magnetic resonance (NMR) can yield detailed structural information of pure substances but requires relatively high analyte concentrations, which can be challenging to obtain for a biological sample.

U.S. Pat. No. 10,832,801, this reference herewith incorporated by reference in its entirety, describes how an IR fingerprint database of mono- and disaccharides can be used to sequence a larger oligosaccharide. In this method, an oligosaccharide is fragmented into mono- and disaccharides, which are then characterized by their m/z and room-temperature IRMPD spectrum. Notably, the method is not sensitive to positional isomers, and it does not include an isomer separation/selection step before or after fragmentation of oligosaccharides. The database consists purely of analytical standards of mono- and disaccharides and cannot be extended to larger species due to the lack of resolution in room-temperature IRMPD spectra.

U.S. Pat. No. 10,359,398, this reference herewith incorporated by reference in its entirety, describes a method for identifying carbohydrate structures based on their m/z ratio and collision cross section (CCS), determined through drift time measurements on negatively charged ions. The identification relies on comparison of determined m/z and CCS values with values stored in a database. The database includes m/z and CCS values of fragments that might be used to identify a compound. The method does not include a strategy to construct the database of m/z and CCS values and therefore relies on the availability of analytical standards. Some types of isomers exhibit extremely similar CCS values, which makes an identification purely based on m/z and CCS value a challenging task, since commercially available instruments are capable to deliver CCS values with an error of 1% or higher.

Accordingly, in light of these deficiencies of the state of the art, strongly improved methods and systems to identify molecules, such as glycans, metabolites, and other biomolecules, and isomers and isobars thereof, are desired, to accelerate the development of small-molecule drugs and biotherapeutics, as well as to facilitate discovery of new biomarkers for disease.

### SUMMARY

According to an aspect of the present invention, a method for identifying and creating a database of molecules is provided. Preferably, the method includes the steps of performing isomer or isobar separation on molecules to obtain separate isomeric or isobaric molecules, measuring mass-to-charge ratios (m/z) to obtain IR fingerprints of the separate isomeric or isobaric molecules, and storing first data on the mass-to-charge ratios (m/z) and/or the IR fingerprints of the separate isomeric or isobaric molecules to a database.

According to another aspect of the present invention, the method further preferably includes the step of identifying unknown molecules, and the step of identifying preferably includes performing isomer or isobar separation on the unknown molecules to obtain separate isomeric or isobaric unknown molecules, measuring mass-to-charge ratios (m/z) to obtain IR fingerprints of the separate isomeric or isobaric unknown molecules, storing second data on the mass-to-charge ratios (m/z) and/or the IR fingerprints of the separate isomeric or isobaric unknown molecules to a memory, and comparing the second data with the first data of the database to identify the unknown molecules.

According to still another aspect of the present invention, the method further includes the steps of identifying unrecorded molecules that have not yet been recorded to the database. In addition, the step of identifying preferably further includes performing isomer or isobar separation on the unrecorded molecules to obtain separate isomeric or isobaric unrecorded molecules, fragmenting the unrecorded molecules into structurally characteristic fragments, the structurally characteristic fragments corresponding to the molecules previously recorded in the database, measuring mass-to-charge ratios (m/z) to obtain IR fingerprints of the structurally characteristic fragments of the unrecorded molecules, storing third data on the mass-to-charge ratios (m/z) and/or the IR fingerprints of the structurally characteristic fragments of the unrecorded molecules to a memory, identifying the structurally characteristic fragments by comparing the third data with the first data on the mass-to-charge ratios (m/z) and/or the IR fingerprints of the database, and determining an original structure of the unrecorded molecules based on the structurally characteristic fragments of the step of identifying, to obtain a new set of recorded molecules.

According to yet another aspect of the present invention, a non-transitory computer-readable medium having computer-readable instructions recorded thereon is provided, the computer-readable instructions configured to perform a method for identifying and creating a database of molecules when executed in a computer that has access to a database. The method preferably includes the steps of controlling a separation device for performing isomer or isobar separation on molecules to obtain separate isomeric or isobaric molecules, instructing the measuring of mass-to-charge ratios (m/z) to obtain IR fingerprints of the separate isomeric or isobaric molecules, and storing data on the mass-to-charge ratios (m/z) and/or the IR fingerprints of the separate isomeric or isobaric molecules to a database.

According to another aspect of the present invention, a system for creating a database of isomers of molecules is provided. Preferably, the system includes a device for performing at least one of ion mobility spectrometry (IMS), liquid chromatography (LC), gas chromatography (GC), and/or capillary electrophoresis (CE) on molecules, a device for performing fragmentation of molecules including at least one of collision-induced dissociation (CID), collision-activated dissociation (CAD), surface-induced dissociation (SID), electron-capture dissociation (ECD), electron-transfer dissociation (ETD), and/or dissociation induced by the absorption of photons, a device for performing infrared (IR) spectroscopic fingerprinting of molecules, and a computing device having access to a database, the computing device configured to record IR fingerprints of separated species in the database, configured to record IR fingerprints and/or mass-to-charge ratios (m/z) of new, structurally characteristic fragments in the database, and/or comparing fragment IR fingerprints to database fingerprints, determining the original structure of the unknown molecules.

We have previously been able to show that a cryogenic, gas-phase, infrared (IR) spectrum of a given molecular ion serves as a unique identifier or molecular fingerprint [1]. When combined with mass spectrometry (MS) and a separation technique, such as high-resolution ion mobility spectrometry (IMS), IR fingerprint spectra of isomer-separated ions can be recorded and stored in a database, together with information about the mass-to-charge ratio (m/z). When a database-compound is encountered in a sample, it can be readily identified based on its mass and IR fingerprint spectrum. If isomers of the compound are present, they can be separated prior to IR fingerprinting with a separation technique. Alternatively, a spectrum of a mixture of isomers can be deconvoluted into the individual database components using an algorithm. Separating isomers prior to IR fingerprinting allows for a more direct way of compound identification and quantification.

The IR fingerprinting technique is suited for molecules with IR active functional groups, such as O—H, N—H, C—H, or C═O groups. The wavelength range that needs to be covered for an IR spectrum to represent a unique molecular fingerprint depends on these IR active sites and their structure-specific intramolecular interaction, e.g., hydrogen-bonding network. For example, U.S. Pat. No. 10,845,337 describes how a cryogenic IR spectrum can be used as a unique molecular fingerprint to identify a compound, this reference herewith incorporated by reference in its entirety. Exemplary data for glycans is given and discussed. The patent does not address how the need for analytical standards can be met and how a database of IR fingerprint spectra can be constructed without such standards. Moreover, U.S. Pat. No. 10,522,337 describes how cryogenic IR fingerprint spectroscopy can be implemented in a high-throughput fashion using a multiplexed approach, this reference herewith incorporated by reference in its entirety. Positional isomer identification, analytical standards, and database construction are not addressed.

The IR fingerprint approach, like other analytical database approaches, requires analytical standards that can be used to add IR spectra and other structure-specific metrics to the database. However, such standards are often either not available in isomerically pure form or their production is not economically viable. Using N-glycans as an example, we developed a novel method to identify positional isomers by measuring and using IR fingerprints of structurally characteristic fragments for which standards are readily available. The method makes use of a fragmentation technique and an optional subsequent IMS separation of fragments before their IR fingerprints are acquired. The focus lies on fragments that are herein referred to as “structurally characteristic fragments.” The structure of such fragments is diagnostic or characteristic for the structure of the precursor molecule and, hence, by determination of the fragment structure, the structure of the precursor molecule structure can also be determined. Once a molecule is identified based on the IR fingerprints of its fragments, data that characterizes or otherwise describes the IR fingerprint of the parent isomer is recorded and stored in the database, for example by the use of a computer that stores database entries into a local or remote memory. This serves two purposes: (1) in a subsequent encounter of this species, it can directly be identified by data of the IR fingerprint without the need to analyze the fragments; and (2) data of the IR fingerprint can be used to identify a larger species whenever the database entry represents a structurally characteristic fragment of that unknown molecule. In addition, isomers identified by the database can serve as precursor structures to create other structurally characteristic fragments that were not accessible through fragmentation of smaller precursor species. Also, the structurally characteristic fragments need not correspond to molecules that exist in solution. According to at least some aspects of the herein presented invention, the method or system can thus serve both to identify isomers of glycans, or other biomolecules including metabolites, and to construct an IR fingerprint database in a bottom-up approach.

The above and other objects, features and advantages of the present invention and the manner of realizing them will become more apparent, and the invention itself will best be understood from a study of the following description with reference to the attached drawings showing some preferred embodiments of the invention.

### BRIEF DESCRIPTION OF THE SEVERAL VIEWS OF THE DRAWINGS

The accompanying drawings, which are incorporated herein and constitute part of this specification, illustrate the presently preferred embodiments of the invention, and together with the general description given above and the detailed description given below, serve to explain features of the invention.

FIG. 1 shows a schematic representation of the newly developed method to identify positional isomers and grow an IR spectroscopic database with minimum necessity for analytical standards;

FIG. 2 shows a schematic representation of the fragment annotation following the Domon and Costello nomenclature [2];

FIG. 3A shows ATD of the singly sodiated glycan standards (m/z 771) after 10.4 m SLIM-IMS separation, FIG. 3B shows Cryogenic IR spectra of standards (m/z 771) Man-2(3) (α-3 configuration) (top and middle panels) and Man-2(6) (α-6 configuration) (bottom panel);

FIG. 4A shows positional isomers of G0-N and their structurally characteristic fragments Man-2(6) and Man-2(3). (b) ATD of G0-N after 10.4 m SLIM-IMS separation, FIG. 4B Cryogenic IR spectra of diagnostic fragments (m/z 771) generated from each peak in the ATD of G0-N (top and middle panel) and a synthetically generated spectrum (bottom panel, dark grey) obtained using a 40:60 contribution of the spectra from peak 1 and peak 2, respectively. The IR fingerprint of the Man-2(6) standard is depicted for comparison (bottom panel, light grey);

FIG. 5A shows possible isomers of the m/z 1136 fragment of G0, FIG. 5B shows ATD of CID-generated m/z 1136 fragments of G0, FIG. 5C shows Cryogenic IR spectra of mobility-separated drift peaks (top-three spectra, light grey) and IR fingerprints of corresponding fragments from the database (dark grey, in background) for comparison with fragment spectra;

FIG. 6A shows a structure of G1 and possible positional isomers of the m/z 1136 fragments after CID, FIG. 6B shows ATD of G1 after four IMS cycles, FIG. 6C shows ATDs of CID-generated Y fragments (m/z 1136) of the two major mobility features of G1, and FIG. 6D shows cryogenic IR fingerprints of mobility-separated drift peaks of m/z 1136 fragments from high- and low-mobility G1 ions, respectively (light grey in foreground). IR reference spectra of corresponding positional isomers from database are shown in dark grey in the background;

FIG. 7 shows exemplary structures of the isobaric or isomeric metabolite molecules used in the proof-of-principle experiments performed herein;

FIG. 8 shows infrared fingerprint spectra of singly sodiated phase II metabolite isomers and isobars, where each spectrum features resolved and distinct absorption lines that are characteristic for the precise molecular identity;

FIGS. 9A and 9B show exemplary results with estradiol glucuronide isomers, with FIG. 9A showing an arrival time distribution of the mixture of the estradiol glucuronide isomers after 20 m (two cycles) of IMS separation, and FIG. 9B shows the IR fingerprint spectra for each of the drift peak in the ATD together with their best-matching database IR fingerprint;

FIGS. 10A and 10B show exemplary results with a mixture of four metabolite isomers, with FIG. 10A showing an arrival time distribution of a mixture of four metabolite isomers after 10 m IMS separation, and FIG. 10B showing IR fingerprints of the individual metabolite drift peaks in the foreground, and their best-matching IR fingerprint from the database for peak 1 and 3, and a synthetic mixture resulting from the spectral deconvolution for peak 2 in the background; and

FIG. 11 shows an exemplary illustration of the one or more steps of the herein described method, and a computer system associated thereto, according to another aspect of the present invention.

### BRIEF DESCRIPTION OF THE SEVERAL EMBODIMENTS

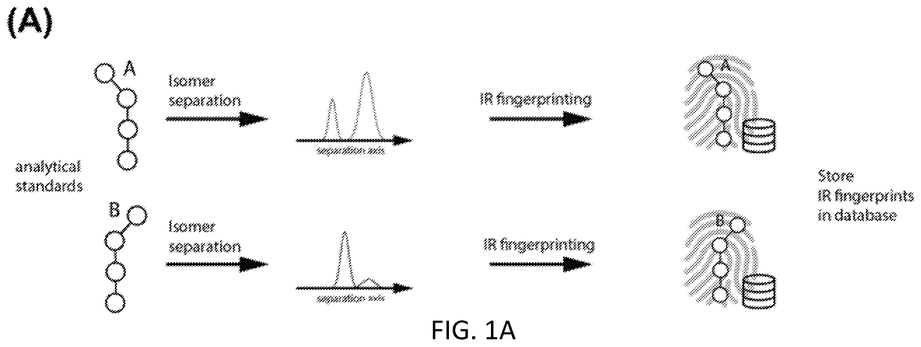

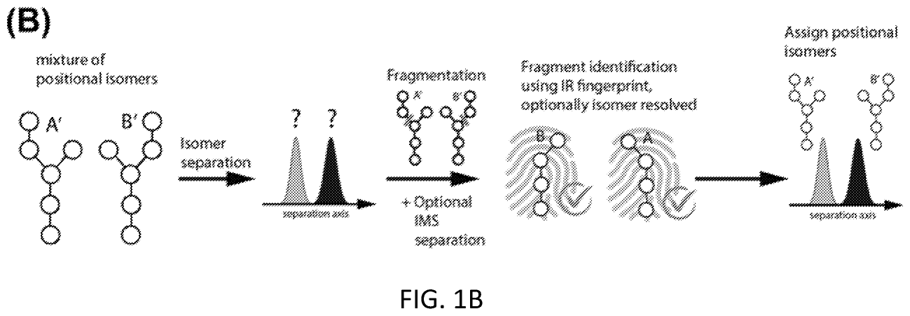

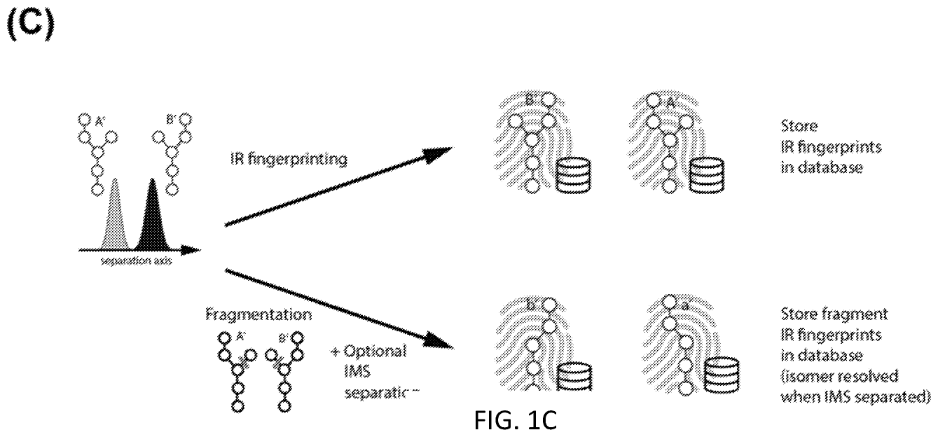

With the representation of FIG. 1, an exemplary flowchart of the method is schematically represented, according to one aspect of the present invention, where the method can include three main steps (A), (B), and (C).
(A) Measurement of IR fingerprints for database:
   i) Perform isomer separation (if necessary, for example but not limited to the use IMS, LC, GC, CE) of pure analytical standards.
   ii) Record or store data on the IR fingerprints of separated species in a database. The IR fingerprint is depicted as a fingerprint pictogram in FIG. 1.
(B) Identification of unknown molecule:
   i) Separate isomers using IMS, LC, GC, CE, or another suitable technique.
   ii) Record or store data on IR fingerprints and compare to existing database entries. If no matching IR fingerprint is found to identify the unknown and unrecorded molecule, continue with the next steps.
   iii) Fragment separated unknown molecules into structurally characteristic fragments (corresponding to the standards in (A) or to other molecules present in the IR fingerprint database).
   iv) Identify fragments using data of the reference IR fingerprints in the IR fingerprint database.
   v) From the structurally characteristic fragment, determine the original structure of the unknown molecule and assign it to the isomeric or isobaric species separated in B(i).
(C) Database extension:
   i) Use the now assigned but unrecorded molecule from (B), record or store data on their IR fingerprints and add them to the database. The newly identified isomers can now be used as structurally characteristic fragments themselves in order to identify larger unknown molecules in a workflow similar to the one described in (B).
   ii) The newly identified molecules might be used as precursor ions to generate new structurally-specific fragments that can then be added to the database as standards. These might be used as standards to identify molecules obtained from solution or those obtained after fragmentation of other molecules.

For example, the method for creating a database of isomers or isobars of molecules as described herein in more detail can include measuring mass-to-charge ratios (m/z) and IR fingerprints for recordation or storage to a database, the measuring including performing isomer/isobar separation of isomeric/isobaric molecules and recording IR fingerprints of separated molecules in a database. Also, the method can further include the step of identifying unknown molecules, the identifying including separating isomers or isobars by a separation technique, fragmenting each separated isomer/isobar into structurally characteristic fragments corresponding to the molecules previously recorded in the database, identifying fragments using the recorded IR fingerprints of the database, determining an original structure and assigning the original structure to the unknown and unrecorded molecules identified in the step of performing and recording, and recording the IR fingerprint of the now identified molecules in the database.

The method can further include a step of measuring m/z and IR fingerprints of new, structurally characteristic fragments from the newly identified molecules, the step of measuring including separating precursor isomers/isobars by a separation technique, fragmenting each separated and previously identified and recorded isomer/isobar into previously unrecorded structurally characteristic fragments, and recording IR fingerprints and m/z of new, structurally characteristic fragments in the database.

In addition, the method can further include a step of separating isomers of the structurally characteristic fragments by ion mobility spectrometry (IMS) prior to recording IR fingerprints and storing in the database. In the context of the embodiments of the present invention, the expression database is to be broadly construed, and can include different types of data structures that can be stored on different types of storage mediums and computer memory, such as but not limited to random access memory (RAM), volatile and non-volatile memory, cloud-based memory, shared local and cloud based memories, hard drives, FLASH based memory, local memory such as cache memories, and the data structure can include but is not limited to file systems, tables, structured query language (SQL), extensible markup language (XML) databases.

Furthermore, the fragmentation of the molecules can be done by different techniques, for example by collision-induced dissociation (CID), for example using the methods, CID devices, and systems described in Unites States application Ser. No. 17/709,446, this reference herewith incorporated by reference in its entirety, collision-activated dissociation (CAD), surface-induced dissociation (SID), electron-capture dissociation (ECD), electron-transfer dissociation (ETD), and dissociation induced by the absorption of photons. Moreover, the spectroscopy measurement can be performed via infrared multiple photon dissociation (IRMPD), the measurement including irradiating molecular ions with photons from a laser source, and detecting the wavelength-dependent fragmentation yield of the precursor ions in a mass spectrometer.

In addition, the spectroscopy measurement can be performed by cryogenic ion spectroscopy, the measurement including storing ions in a cryogenic ion trap, cooling ions by collisions with a cold buffer gas, tagging cold ions by one or multiple tag molecules, such as nitrogen, irradiating ions with photons from a laser source, recording the wavelength-dependent depletion of tagged cold ions using a mass spectrometer. Moreover, the spectroscopy can be performed at photon energies preferably ranging from 500 cm⁻¹ to 4000 cm⁻⁴, for instance more preferably between 3250 cm⁻¹ to 3750 cm⁻¹.

In addition, preferably the molecules are selected from a list comprising oligosaccharides (glycans), polypeptides, nucleic acids, lipids, primary metabolites, and secondary metabolites. Furthermore, the step of separating can be performed by at least one of ion mobility spectrometry (IMS), liquid chromatography (LC), gas chromatography (GC), or capillary electrophoresis (CE).

### EXAMPLES

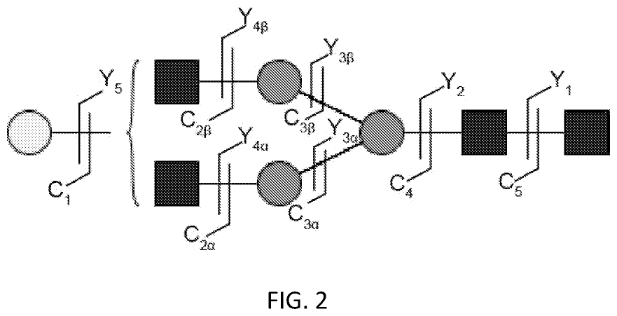

In an exemplary variant, the method was applied to a series of positional isomers of N-glycans, according to an aspect of the present invention. Glycan notation follows the one issued by the Consortium for Functional Glycomics (CFG). Nomenclature of glycan fragments is according to Domon/Costello and is summarized in FIG. 2. In addition, in another exemplary variant, the method was applied to a collection of isomeric and isobaric phase II metabolite molecules.

The analysis was performed using a home-built electrospray ionization ultrahigh-resolution ion mobility spectrometer coupled to a cryogenic ion-trap and a time-of-flight (TOF) MS. The instrument follows a design that was previously described in reference [3]. The mobility separation was performed using a cyclic IMS device which makes use of traveling-wave IMS and reaches a resolving power of ˜200 after a single separation cycle. The overall resolving power can be increased by a factor of \sqrt{n} as the number of separation cycles n is increased. Cyclic IMS was implemented using structures for lossless ion manipulation (SLIM) developed in the Smith group at PNNL [4]. After IMS separation, ions can be introduced into a separate region that was designed to perform CID [5], see also Unites States application Ser. No. 17/709,446, this reference herewith incorporated by reference in its entirety. It is situated within the IMS region, which allows to reintroduce CID fragments for further IMS separation. To record cryogenic infrared spectra, ions are guided towards a cryogenic ion trap, held at a temperature of 45 K, where they are cooled, trapped, and tagged with N₂ molecules prior to being irradiated by an infrared (IR) laser beam. Nitrogen-tagged ions that absorb an IR photon of a wavelength that is resonant with a molecular vibration will lose the N₂ tag molecule. Lastly, ions are extracted towards a TOF mass analyzer to measure their mass-to-charge (m/z) ratio. The cryogenic IR fingerprint of the considered ions is obtained by monitoring the depletion of the N₂-tagged ions as a function of the laser wavelength.

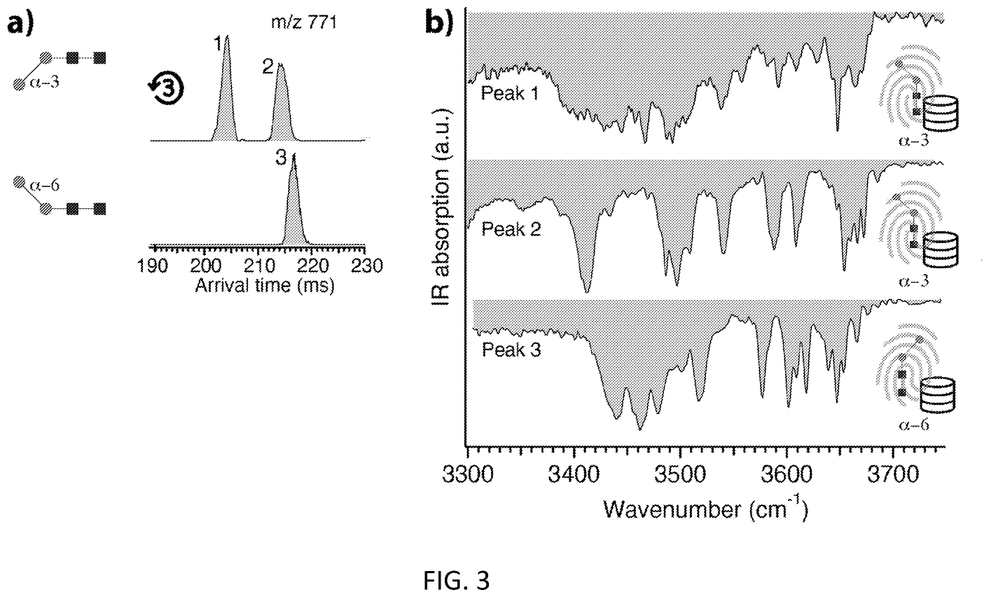

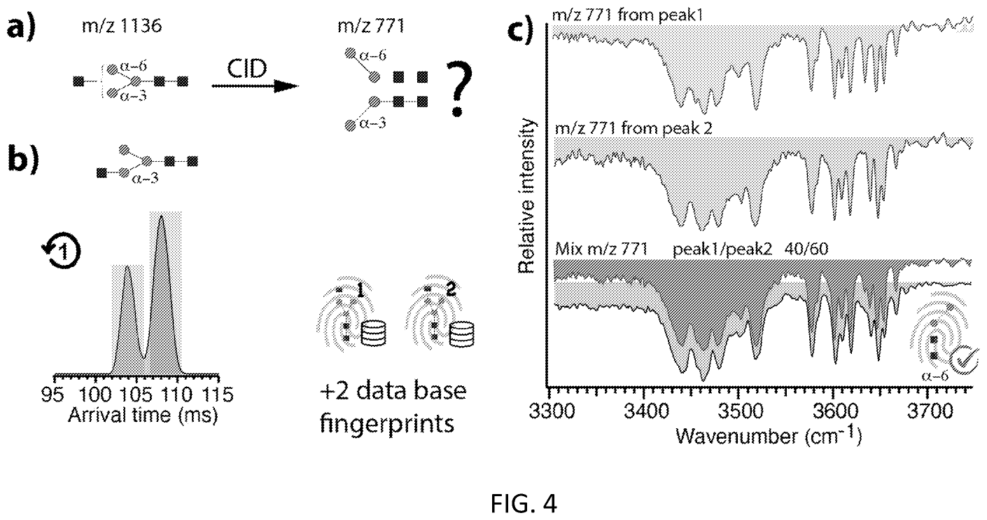

With respect to the identification of glycan positional isomers, following the protocol described above, it is possible to identify the regio- or positional isomers of G0-N that differ in the position of a terminal monosaccharide GlcNAc, which can either be attached to the α-3 or the α-6 branch. We first determined the isomeric fragments Y_{3α} and Y_{3β} (m/z 771 singly sodiated) generated by CID as being structurally characteristic fragments for the positional isomers G0-N(3) and G0-N(6), respectively, see FIG. 4A showing that the two isomeric fragments can be characteristic for one or the other positional isomers of the glycan G0-N. An important premise on which we base our identification approach is that the observed fragments have a higher propensity for the dissociation of a single covalent bond rather than the dissociation of multiple bonds. To test this hypothesis, we purchased a pair of molecules that correspond to the structurally characteristic fragments Y_{3α} and Y_{3β} and measured their isomer-specific IR fingerprints prior to adding data that characterizes these fingerprints to the database, for example using a general-purpose computer that stores the data on the fingerprints to a database which is stored in the local memory, or a memory that is accessed via a network, these molecules being depicted in FIG. 3A. An IMS-resolved isomer distribution of the two analytical standards in their singly sodiated form after three (3) separation cycles is depicted in FIG. 3A. While two isomers are observed for the α-3 isomer, only one could be resolved for the α-6 isomer. The two species observed for the α-3 isomer might be a result of the two and β anomers of the reducing end that every glycan with a free reducing end can form. Since only one IMS peak is observed for the α-6 species, the resulting IR fingerprint spectrum must represent a superposition of these two α and β reducing-end anomers. Nevertheless, the obtained IR spectra shown in FIG. 3B can serve as identifiers for their respective structures.

Following step (B) in the workflow described above, a sample containing an unknown mixture of G0-N positional isomers (purchased from TheraProteins) was ionized to yield singly sodiated species, mobility-separated, and fragmented using CID. FIG. 4B shows the arrival time distribution (ATD) of the singly sodiated G0-N ions (m/z 1136) after one IMS separation cycle. The ATD features two mobility peaks which were fragmented individually to produce the 771 m/z structurally characteristic fragments as indicated in FIG. 4A, and their IR fingerprints were recorded individually (top and middle spectra in FIG. 4C).

In the bottom panel of FIG. 4C, the IR fingerprint of the α-6 standard (light grey) is compared to a synthetic 40:60 mixture (dark grey) of the IR fingerprints corresponding to the 771 m/z fragments produced from peak 1 and peak 2 of G0-N. This is equivalent to a spectrum of the 771 m/z fragments without IMS separation, i.e., without separating the α and β reducing-end anomers. We confirm by visual comparison that the fingerprints of the mixture and that of the depicted database fingerprint are virtually identical, indicating that both mobility peaks of G0-N correspond to the α-3 positional isomer, presumably the α- and β anomers that coexist in solution. Consequently, and against our expectations, only one possible positional isomer of G0-N was present in the unknown mixture. This was now identified to be the isomer with the terminal GlcNAc monosaccharide attached onto the α-3 branch (G0-N(3)). While it was not possible to resolve these reducing-end anomers for the α-6 standard with IMS, it was possible to do so for the G0-N(3) positional isomer. As a final step, the IR fingerprints of the now identified isomers of G0-N(3) are recorded and stored in the database, which is thereby extended by two new entries.

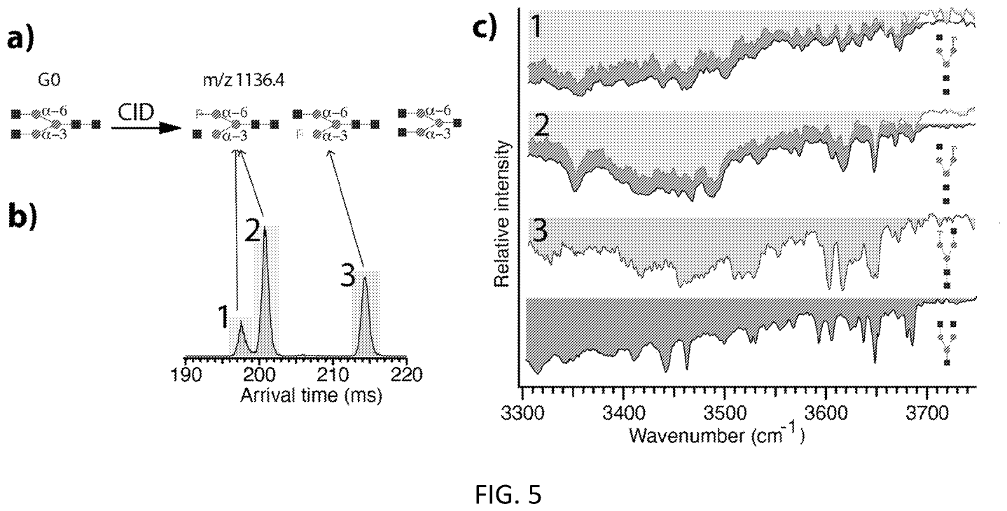

In the following section, it is demonstrated that the use of the workflow of the method described above to generate an IR fingerprint for G0-N(6) is possible without the need for an isomerically pure analytical standard. In a first step we performed CID on the glycan G0 without prior mobility separation, see FIG. 5A. The fragment with m/z 1136 generated upon the loss of a GlcNAc monosaccharide can exist in three isomeric forms (Y_{4α}, Y_{4β}, and C₄) where Y_{4α} and Y_{4β} correspond to the molecules G0-N(6) and G0-N(3), respectively. An additional isomer corresponding to the C₄ fragment can be purchased as a standard.

Following CID, the m/z 1136 fragments were mobility separated. The resulting ATD of the fragments, displayed in FIG. 5B, shows three mobility peaks indicating the presence of different isomers produced upon CID.

IR spectra of all three mobility peaks were recorded, shown in FIG. 5C, light grey in foreground, and compared to the two IR fingerprints of G0-N(3) obtained previously as well as to an IR fingerprint of the additional standard corresponding to the C₄ fragment (FIG. 5C, dark grey in background). The IR spectra of first two mobility peaks are virtually identical to those of the two G0-N(3) isomers, i.e., these two peaks correspond to the Y₄a fragment from G0 (presumably the α- and β anomers). The IR fingerprint of the third ATD feature does not correspond to the reference fingerprint of the hypothetical C₄ fragment and, consequently, the third ATD peak can be assigned to the Y₄a fragment by exclusion. Its IR fingerprint can now be stored in the database as a reference for the G0-N(6) glycan. Now that we have identified the IR fingerprints of the G0-N(3) and G0-N(6) glycans, they can not only be used for their identification in complex samples, but also as structurally characteristic fragments to identify positional isomers of even larger glycans. We demonstrate this below using G1 as an example.

The terminal galactose in the glycan G1 can either be linked to the α-3 or α-6 branch, leading to the positional isomers G1(3) and G1(6), respectively. The structurally characteristic CID fragments that can be used to identify G1-positional isomers G1(3) and G1(6) are Y_{4α} and Y_{4β}, respectively, and correspond to the two positional isomers G0-N(3) and G0-N(6) that we identified above and added to the IR fingerprint database, see FIG. 6A.

Singly sodiated G1 ions were mobility separated then fragmented by CID. An ATD of G1 after six IMS separation cycles is shown in FIG. 6B. CID was performed on the two main mobility features at 430 ms and 490 ms separately, followed by their IR spectroscopic fingerprinting. An additional IMS separation was performed to separate possible positional isomers of the m/z 1136 fragments, and the ATDs of the fragments originating from the two different mobility features of G1 are shown in FIG. 6C. Two species can be observed for fragments originating from the higher-mobility ions of G1, and a single ATD peak is observed for fragments from the lower-mobility ions of G1.

Subsequently, the IR spectra of the three different fragment isomers were recorded (FIG. 6D, light grey in foreground) and compared to spectra from the IR fingerprint database (dark grey in background). Visual comparison of the fingerprint spectra confirms that the fragments from the higher-mobility G1 ions are identical to the two G0-N(3) isomers, while those from the lower-mobility G1 ions can be identified as G0-N(6). Consequently, based on structurally characteristic fragment identification, we can assign the first peak of the ATD of G1 to its positional isomer G1(6) and the second mobility region (two not fully resolved features) to the positional isomer G1(3). As before, the IR fingerprint database can now be extended by adding the IR fingerprints of the identified positional isomers of G1, which will serve identification of larger glycan structures.

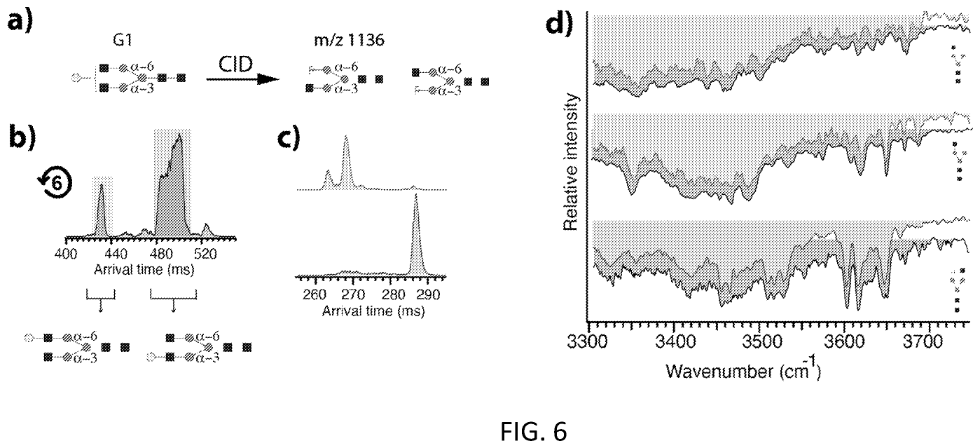

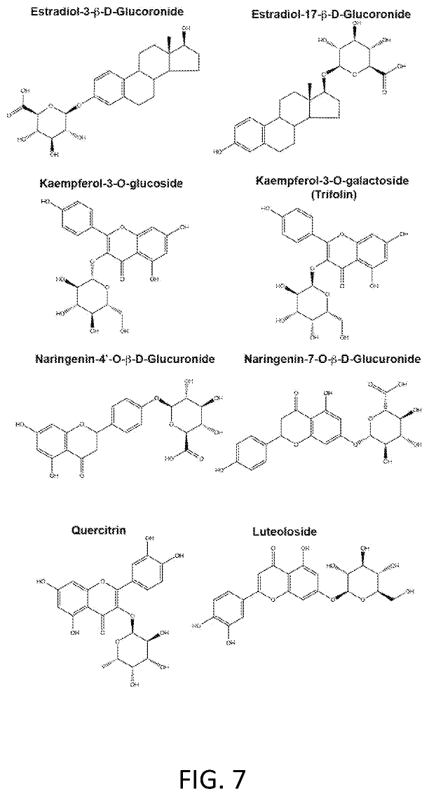

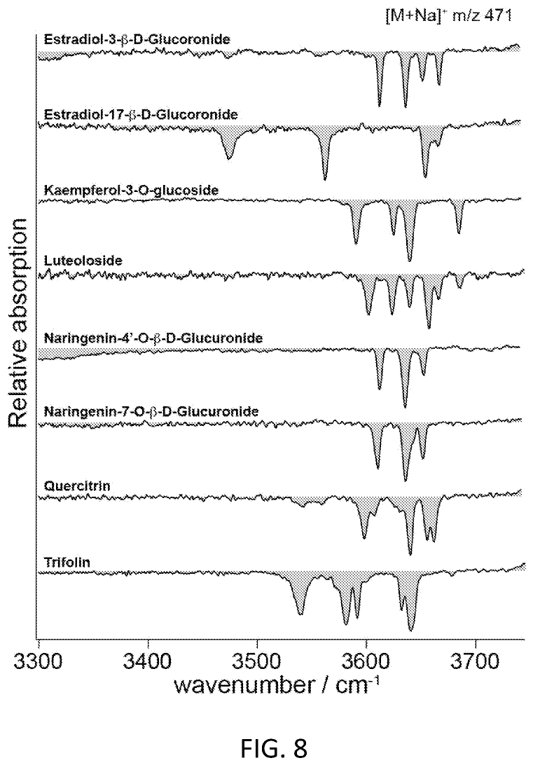

With respect to the identification of metabolite isomers and isobars, in contrast to LC-MS/MS workflows that are currently used to separate and identify metabolite isomers and isobars, according to one aspect of the present invention, the method described herein can use high-resolution IMS for rapid isomer separation and cryogenic IR fingerprint spectroscopy for a confident and reproducible identification, and does not require recurrent calibration with analytical standards. In such method, the initial IR fingerprint database can be built or established for a set of (8) eight metabolite isobars/isomers, as illustrated in FIG. 7, in positive and negative MS modes according to step A in the identification scheme discussed herein. The set of molecules includes two (2) estrogen metabolite positional isomers estradiol-3-β-D-glucuronide and etradiol-17-β-D-glucuronide. The remaining six (6) isomeric and/or isobaric metabolites are flavonoids originating from a variety of plants and are composed as follows: kaempferol-3-O-glucoside and kaempferol-3-O-galactoside where the monosaccharides attached to the kaempferol core are isomeric. Naringenin-4′-O-β-glucuronide and naringenin-7-O-β-glucuronide which are positional isomers of each other. Quercitrin and trifolin differ in their core and as well as in the attached glycan. To build the initial IR spectral fingerprint database, the metabolite standards were analyzed separately. The IR fingerprints of singly sodiated species recorded in positive ion mode and are shown in FIG. 8. Each database IR spectrum was recorded in the wavenumber region from 3300 cm⁻¹ to 3750 cm⁻¹. Each of these reference spectra were measured within sixty (60) seconds during which the laser was scanned over the 450 cm⁻¹ range. Each absorption was oversampled during the acquisition to ensure optimum signal-to-noise as well as to achieve maximum spectral resolution.

Several well-resolved absorption bands can be observed, originating from relatively free OH oscillators in the region between 3550 cm⁻¹-3700 cm⁻¹ as well as more hydrogen-bonded OH oscillators below 3550 cm⁻¹. Although the structures of the considered metabolites are, in some cases, extremely similar, the recorded spectra are each unique and highly structured, which makes them ideal fingerprints. For the current set of metabolites, it can also be observed that the region 3500 cm⁻¹-3700 cm⁻¹ would be sufficient for a positive identification as it includes most of the absorption bands. Spectra of the negatively charged or singly deprotonated species were recorded as well and feature similarly unique absorption bands (data not shown).

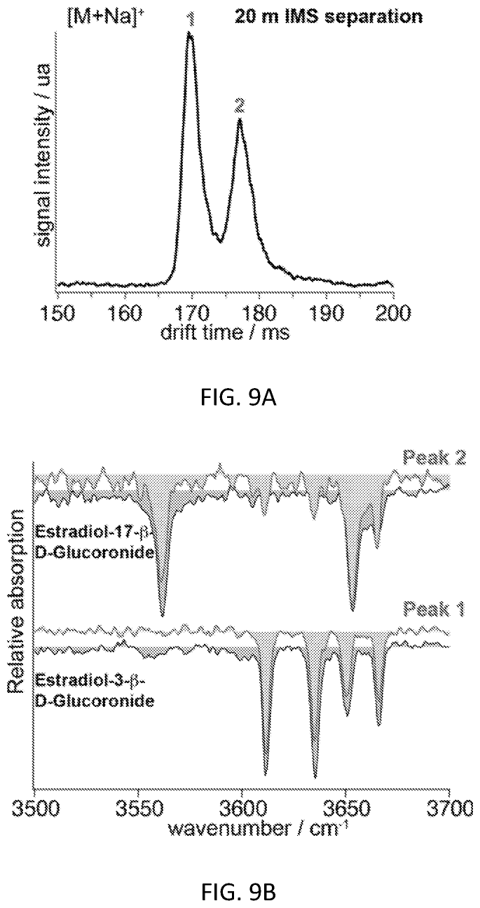

In the following paragraphs, a description is provided for applying step B of the identification method to identify individual components in mixtures of metabolite isomers. The first mixture tested here contains both estradiol glucuronide isomers mentioned in the previous section. The isomers estradiol-3-β-D-glucuronide and etradiol-17-β-D-glucuronide result from different metabolic pathways, which are dependent on the concentration of certain enzymes in the liver and kidneys. The arrival time distribution obtained after two IMS separation cycles (20 m drift path) is shown in FIG. 9A. We observe two ion mobility peaks, one per isomer present in the mixture. After mobility separation, IR fingerprints were recorded using a 10-second long spectral acquisition scheme. A comparison between the fingerprints obtained from the mixture and the database entries for the estradiol glucuronide isomers is shown in FIG. 9B. A visual comparison of the IR fingerprints clearly identifies the first mobility peak to estradiol-3-β-D-glucuronide and the second mobility peak to estradiol-17-β-D-glucuronide.

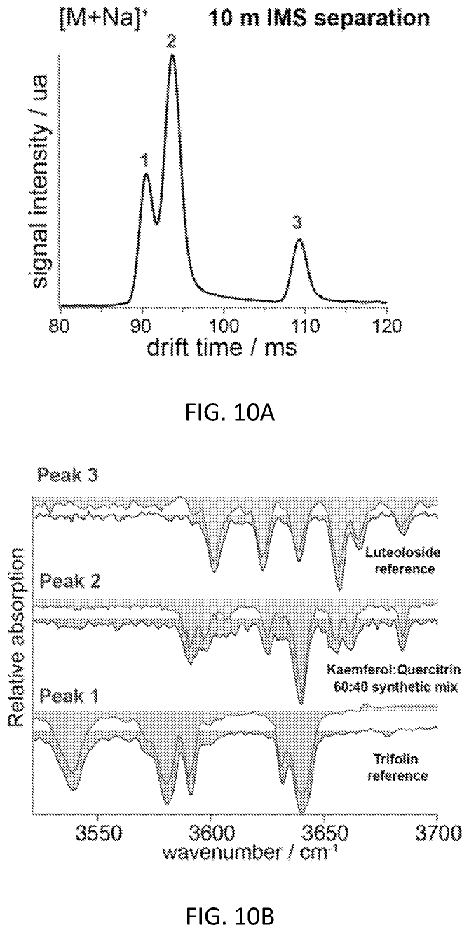

A second more complex metabolite mixture was composed of the four (4) components kaempferol-3-O-glucoside, kaempferol-3-O-galactoside (trifolin), quercitrin, and luteoloside. The ATD obtained after 10 m IMS separation, displayed in FIG. 10A, includes three distinct mobility peaks. As the mixture is composed by four components of with only two seem to be fully resolved, one of the mobility peaks must contain two unresolved metabolites. Following our IR fingerprinting method according to step B, we can assign peak one and three to trifolin and luteoloside, respectively, based on a visual comparison of the spectra acquired from the mixture to database fingerprints displayed in FIG. 10B top and bottom. The spectrum obtained for the second drift peak contains features from both of the two elusive isomers quercitrin and kaempferol-3-O-glucoside. While most features from the quercitrin IR fingerprint can be easily identified, an absorption at 3380 cm¹ and other details in the spectrum indicate the presence of kaempferol-3-O-glucoside. A fitting algorithm using the IR fingerprints of the eight isomeric and isobaric molecules as a basis set can be applied to obtain a synthetic IR fingerprint with the components kaempferol-3-O-glucoside and quercitrin at a ratio of 60/40 to match the experimentally obtained spectrum of the second drift peak as shown in the middle panel of FIG. 10B. See for example Abikhodr et al., “Identifying Mixtures of Isomeric Human Milk Oligosaccharides by the Decomposition of IR Spectral Fingerprints,” Analytical Chemistry Vol. 93, No. 44, year 2021, pp. 14730-14736, this reference herewith incorporated by reference in its entirety. The second drift peak can therefore be identified as containing a mixture of kaempferol-3-O-glucoside and quercitrin. This example illustrates the analytical power of IR fingerprinting even when a separation of isomeric or isobaric compounds is not possible. In the case of the considered metabolites, IR fingerprinting allows to confidently identify all present species, while even the IMS technology offering the highest IMS resolving power available today fails to do so.

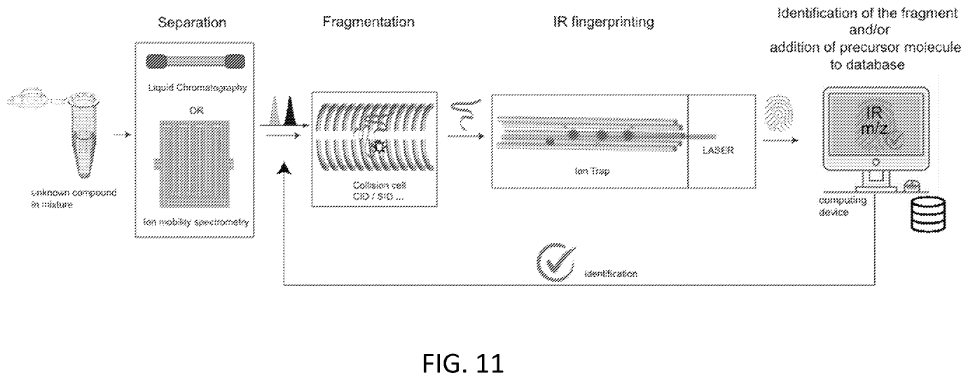

FIG. 11 shows an exemplary illustration of the one or more steps of the herein described method, and a computer system associated thereto, according to another aspect of the present invention, showing a schematic representation of a device for performing at least one of ion mobility spectrometry (IMS), liquid chromatography (LC), gas chromatography (GC), and/or capillary electrophoresis (CE) on molecules, a device for performing fragmentation of molecules including at least one of collision-induced dissociation (CID), collision-activated dissociation (CAD), surface-induced dissociation (SID), electron-capture dissociation (ECD), electron-transfer dissociation (ETD), and/or dissociation induced by the absorption of photons, a device for performing infrared (IR) spectroscopic fingerprinting of molecules, and a computing device having access to a database, the computing device configured at least to record IR fingerprints of separated species in the database, configured to record IR fingerprints and m/z of new, structurally characteristic fragments in the database, and/or comparing fragment IR fingerprints to database fingerprints, determining the original structure of the unknown molecules.

According to some aspects of the present invention, the herein discussed cryogenic IR fingerprint method requires analytical standards that can be used to build an initial database, for example a few standards of the smallest molecular building blocks, in other words standards that correspond to characteristic fragments of larger molecules. In the case of glycans, isomerically pure standards of different positional isomers are often unavailable or expensive. According to some aspects of the invention, a method is provided to build a database in a bottom-up manner, using only few commercial analytical standards and isomeric fragments that are specific to a particular isomeric precursor ion. The approach to use structurally characteristic fragments for identification in conjunction with cryogenic IR spectroscopy has not previously been reported or protected. The alternative to the method described here is to use purified commercial or synthetic standards to record reference IR spectra for each isomeric species that is in question. Without the herein presented method and system, a database of IR spectra could only be constructed using commercial analytical standards, if they are available.

IR spectra of larger glycans, for example glycans including ten (10) or more monosaccharides, have proven to be sufficiently characteristic, i.e., they provide enough information for a compound to be unambiguously identified. This allows to build a database towards larger species. Different fragmentation channels cannot easily be controlled, however, glycan fragmentation often yields structurally characteristic fragments like the ones described in the methods applied here. Fragments can often yield isomeric structures. In the case of N-glycans, our research showed that a fragmentation channel involving cleavage of a single covalent bond is always favored over one that involves multiple bond cleavages. This proves to be advantageous in the assignment of fragments using m/z information. The fact that the structure, and hence the IR fingerprint spectrum, of CID fragments of oligosaccharides can correspond to those of intact glycans ionised from solution is not a priori obvious and was thoroughly investigated and confirmed in our laboratory.

It is noted that cryogenic gas-phase IR spectroscopy cannot be performed using commercial instrumentation and its implementation into a mass-spectrometer type instrument presents a challenge. There is no commercial instrument to perform the experiments described here. We made these measurements on a custom-designed instrument.

The ability to perform ion fragmentation after isomer separation, followed by another isomer-separation step and subsequent IR spectroscopic analysis is technically challenging and to date has only been done using our custom-designed instrument.

According to one embodiment of the present invention, a workflow that was used to identify regio-isomers of N-glycans, in a custom-built instrument that provides means for isomer separation (IMS) and ion fragmentation (CID) and includes a cryogenic ion trap to record IR fingerprint spectra using a tunable LASER source and a mass analyzer. The separation method used was based on structures for lossless ion manipulation (SLIM) [4]. The CID method was based on SLIM technology but developed in our laboratory [5].

In another embodiment, the method was applied to build a database of IR fingerprint spectra from larger molecules. The instrument required for this embodiment provides the means for initial isomer separation by IMS, ion fragmentation (CID), fragment isomer separation using IMS, and cryogenic IR spectroscopic fingerprinting of the separated isomers using a tunable LASER source and a mass analyzer.

In another embodiment, the IR fingerprint spectra can be stored in a database on a computing device. The step of comparison of IR fingerprint spectra obtained from unknown isomers to database IR fingerprints is performed using an algorithm. Upon previously defined conditions, the algorithm automatically determines if a spectrum of an unknown compound is present in the database or not. Specifically, we have successfully implemented the method of principle component analysis (PCA) to perform the step of comparison between database spectra and those of unknowns. In addition, we applied PCA to evaluate the uniqueness of different wavenumber regions of database IR fingerprints, i.e., an algorithm can evaluate the wavenumbers that a fingerprint spectrum needs to be recorded in order to identify an isomer with sufficient confidence.

Potential fields of applications of the herein presented methods and systems are for example but not limited to glycomics (biomarker research, characterization and process control of biotherapeutics, characterization of milk and other food oligosaccharides), and metabolomics (identification of metabolites).

According to some aspects of the herein presented method and system, the method can become available in research and industry either through development of a commercially available version of our instrument or though partnering with an existing scientific instrument manufacturer to jointly develop an instrument that allows the measurements described here. Potential developers/producers are existing scientific instrument manufacturers. Potential users could be and are not limited to pharmaceutical companies, analytical service companies, biomedical research laboratories, and university and government research laboratories.

While the invention has been disclosed with reference to certain preferred embodiments, numerous modifications, alterations, and changes to the described embodiments, and equivalents thereof, are possible without departing from the sphere and scope of the invention. Accordingly, it is intended that the invention not be limited to the described embodiments, and be given the broadest reasonable interpretation in accordance with the language of the appended claims.

### REFERENCES

[1] C. Masellis, N. Khanal, M. Kamrath, D. E. Clemmer, T. R. Rizzo, “Cryogenic vibrational spectroscopy provides unique fingerprints for glycan identification”, J. Am. Soc. Mass Spectrom. 2017, 28, 2217-2222.
[2] Domon, B.; Costello, C. E., “A Systematic Nomenclature for Carbohydrate Fragmentations in FAB-MS/MS Spectra of Glycoconjugates”, Glycoconjugate J. 1988, 5, 397-409.
[3] A. Ben Faleh, S. Warnke, T. R. Rizzo, “Combining ultrahigh-Resolution ion-mobility spectrometry with cryogenic infrared spectroscopy for the analysis of glycan mixtures”, Anal. Chem. 2019, 91, 4876-4882.
[4] L. Deng et al., “Serpentine Ultralong Path with Extended Routing (SUPER) High Resolution Traveling Wave Ion Mobility-MS using Structures for Lossless Ion Manipulations”, Anal. Chem. 2017, 89, 4628-4634.
[5] P. Bansal, V. Yatsyna, A. H. AbiKhodr, S. Warnke, A. Ben Faleh, N. Yalovenko, V. H. Wysocki, T. R. Rizzo, “Using SLIM-based IMS-IMS together with cryogenic infrared spectroscopy for glycan analysis”, Anal. Chem. 2020, 92, 9079-9085.

### KEY WORDS

IR spectroscopy, IR fingerprinting, glycan analysis, isomer identification

1-18. (canceled)

19. A method for identifying, and creating a database of, molecules, comprising the steps of:
performing isomer or isobar separation on molecules to obtain separate isomeric or isobaric molecules;
measuring mass-to-charge ratios (m/z) through mass spectrometry and performing infrared (IR) spectroscopy to obtain IR fingerprints of the separate isomeric or isobaric molecules;
storing first data on the mass-to-charge ratios (m/z) and the IR fingerprints of the separate isomeric or isobaric molecules to a database, thereby obtaining recorded molecules; and
identifying unrecorded molecules that have not yet been recorded to the database, the step of identifying including,
performing isomer or isobar separation on the unrecorded molecules to obtain separate isomeric or isobaric unrecorded molecules,
fragmenting the unrecorded molecules into structurally characteristic fragments, the structurally characteristic fragments corresponding to the molecules previously recorded in the database,
measuring mass-to-charge ratios (m/z) through mass spectrometry and performing infrared (IR) spectroscopy to obtain IR fingerprints of the structurally characteristic fragments of the unrecorded molecules,
storing second data on the mass-to-charge ratios (m/z) and the IR fingerprints of the structurally characteristic fragments of the unrecorded molecules to a memory,
identifying the structurally characteristic fragments by comparing the second data with the first data on the mass-to-charge ratios (m/z) and the IR fingerprints of the database,
determining an original structure of the unrecorded molecules based on the structurally characteristic fragments of the step of identifying, to obtain a new set of recorded molecules.

20. The method of claim 19, further comprising the step of:
storing third data on the mass-to-charge ratios (m/z) and the IR fingerprints of the new set of recorded molecules to the database, after the step of determining.

21. The method of claim 20, further comprising the step of:
identifying unknown molecules, the step of identifying including,
performing isomer or isobar separation on the unknown molecules to obtain separate isomeric or isobaric unknown molecules,
measuring mass-to-charge ratios (m/z) through mass spectrometry and performing infrared (IR) spectroscopy to obtain IR fingerprints of the separate isomeric or isobaric unknown molecules,
storing fourth data on the mass-to-charge ratios (m/z) and the IR fingerprints of the separate isomeric or isobaric unknown molecules to a memory, and
comparing the fourth data with the third data of the database to identify the unknown molecules.

22. The method of claim 19, further comprising the step of:
performing isomer or isobar separation on the new set of recorded molecules from the step of determining, to obtain separate isomeric or isobaric species of the new set of recorded molecules;
fragmenting the new set of recorded molecules into structurally characteristic fragments that were not previously recorded;
measuring mass-to-charge ratios (m/z) through mass spectrometry and performing infrared (IR) spectroscopy to obtain IR fingerprints of the structurally characteristic fragments of the new set of recorded molecules; and
storing fifth data on the mass-to-charge ratios (m/z) and the IR fingerprints of the structurally characteristic fragments of the new set of recorded molecules to the database.

23. The method of claim 19, further comprising the step of:
performing isomer or isobar separation on molecules serving as an analytical standard, to obtain separate isomeric or isobaric molecules serving as the analytical standard;
fragmenting the separate isomeric or isobaric molecules into structurally characteristic fragments that were not previously recorded;
measuring mass-to-charge ratios (m/z) through mass spectrometry and performing infrared (IR) spectroscopy to obtain IR fingerprints of the structurally characteristic fragments of the separate isomeric or isobaric molecules; and
storing sixth data on the mass-to-charge ratios (m/z) and the IR fingerprints of the structurally characteristic fragments of the separate isomeric or isobaric molecules, to include data on fragments of the analytical standards to the database.

24. The method of claim 19, further comprising the step of:
performing isomer or isobar separation of the structurally characteristic fragments, after the step of fragmenting, the performing done by ion mobility spectrometry (IMS).

25. The method of claim 19 wherein the step of fragmenting includes at least one of collision-induced dissociation (CID), collision-activated dissociation (CAD), surface-induced dissociation (SID), electron-capture dissociation (ECD), electron-transfer dissociation (ETD), and/or dissociation induced by the absorption of photons.

26. The method of claim 19, wherein the step of measuring includes infrared multiple photon dissociation (IRMPD), irradiating molecular ions with photons from a laser source, and monitoring and recording the wavelength-dependent fragmentation yield of the precursor ions using a mass spectrometer.

27. The method of claim 19, wherein the step of measuring includes cryogenic ion spectroscopy, the cryogenic ion spectroscopy including the steps of
storing ions in a cryogenic ion trap;
cooling ions by collisions with a cold buffer gas;
tagging cold ions by one or multiple tag molecules;
irradiating ions with photons from a laser source; and
recording the wavelength-dependent depletion of tagged cold ions using a mass spectrometer.

28. The method of claim 19, wherein the step of measuring includes spectroscopy at photon energies ranging from 500 cm⁻¹ to 4000 cm⁻⁴.

29. The method of claim 19, wherein the step of measuring includes spectroscopy at photon energies ranging from 3250 cm⁻¹ to 3750 cm⁻¹.

30. The method of claim 19, wherein the molecules are selected from a list comprising at least one of oligosaccharides, glycans, polypeptides, nucleic acids, lipids, primary metabolites, and/or secondary metabolites.

31. The method of claim 19, wherein the step of performing isomer or isobar separation includes at least one of ion mobility spectrometry (IMS), liquid chromatography (LC), gas chromatography (GC), and/or capillary electrophoresis (CE).

32. A non-transitory computer-readable medium having computer-readable instructions recorded thereon, the computer-readable instructions configured to perform a method for identifying, and creating a database of, molecules when executed in a computer that has access to a database, the method including the steps of:
controlling a separation device for performing isomer or isobar separation on molecules to obtain separate isomeric or isobaric molecules;
instructing the measuring of mass-to-charge ratios (m/z) through mass spectrometry and the performing infrared (IR) spectroscopy to obtain IR fingerprints of the separate isomeric or isobaric molecules;
storing first data on the mass-to-charge ratios (m/z) and the IR fingerprints of the separate isomeric or isobaric molecules to a database, thereby obtaining recorded molecules; and
identifying unrecorded molecules that have not yet been recorded to the database, the step of identifying including,
instructing the performing of isomer or isobar separation on the unrecorded molecules to obtain separate isomeric or isobaric unrecorded molecules,
controlling a fragmentation device for fragmenting the unrecorded molecules into structurally characteristic fragments, the structurally characteristic fragments corresponding to the molecules previously recorded in the database,
instructing the measuring of mass-to-charge ratios (m/z) through mass spectrometry and the performing infrared (IR) spectroscopy to obtain IR fingerprints of the structurally characteristic fragments of the unrecorded molecules,
storing second data on the mass-to-charge ratios (m/z) and the IR fingerprints of the structurally characteristic fragments of the unrecorded molecules to a memory,
identifying the structurally characteristic fragments by comparing the second data with the first data on the mass-to-charge ratios (m/z) and the IR fingerprints of the database, and
determining an original structure of the unrecorded molecules based on the structurally characteristic fragments of the step of identifying, to obtain a new set of recorded molecules.

33. The non-transitory computer-readable medium of claim 32, wherein the method further comprises the step of:
storing third data on the mass-to-charge ratios (m/z) and the IR fingerprints of the new set of recorded molecules to the database, after the step of determining.

34. The non-transitory computer-readable medium of claim 33, wherein the method further comprises the step of:
identifying unknown molecules, the step of identifying including,
instructing the performing of isomer or isobar separation on the unknown molecules to obtain separate isomeric or isobaric unknown molecules,
instructing the measuring of mass-to-charge ratios (m/z) through mass spectrometry and the performing infrared (IR) spectroscopy to obtain IR fingerprints of the separate isomeric or isobaric unknown molecules,
storing fourth data on the mass-to-charge ratios (m/z) and the IR fingerprints of the separate isomeric or isobaric unknown molecules to a memory, and
comparing the fourth data with the third data of the database to identify the unknown molecules.

35. A system for creating a database of isomers of molecules, comprising:
a device for performing at least one of ion mobility spectrometry (IMS), liquid chromatography (LC), gas chromatography (GC), and/or capillary electrophoresis (CE) on molecules;
a device for performing fragmentation of molecules including at least one of collision-induced dissociation (CID), collision-activated dissociation (CAD), surface-induced dissociation (SID), electron-capture dissociation (ECD), electron-transfer dissociation (ETD), and/or dissociation induced by the absorption of photons;
a device for performing infrared (IR) spectroscopic fingerprinting of molecules; and
a computing device having access to a database, the computing device configured to record IR fingerprints of separated species in the database, configured to record IR fingerprints and mass-to-charge ratios (m/z) of new, structurally characteristic fragments in the database, and/or comparing fragment IR fingerprints to database fingerprints, determining the original structure of the unknown molecules.
